(19) United States
(12) Patent Application Publication
(10) Pub. No.: US 2026/0125726 A1
(43) Pub. Date: May 7, 2026

(54) METHOD FOR THE ENZYMATIC PRODUCTION OF SOLUBLE FIBRES

(71) Applicant: ROQUETTE FRERES, LESTREM (FR)

(72) Inventors: Pierre LANOS, LA BASSEE (FR); Matthieu RAMETTE, ARRAS (FR); Magali REMAUD-SIMEON, RAMONVILLE-SAINT-AGNE (FR); Claire MOULIS, VIEILLEVIGNE (FR); Sandra PIZZUT-SERIN, MAS-SAINTES-PUELLES (FR); Etienne SEVERAC, PIBRAC (FR)

(21) Appl. No.: 18/881,587

(22) PCT Filed: Jul. 10, 2023
(86) PCT No.: PCT/FR2023/051068

§ 371 Date: Dec. 22, 2025

(30) Foreign Application Priority Data
Jul. 11, 2022 (FR) FR2207071

(51) Int. Cl. C12P 19/04; C12N 9/10; C12P 19/18

(52) U.S. Cl. CPC C12P 19/04; C12N 9/1051; C12N 9/107; C12P 19/18; C12Y 204/01005; C12Y 204/01018

(57) ABSTRACT

The invention relates to a method for preparing a mixture of poorly-digestible alpha-glucans from a substrate rich in oligosaccharides having a degree of polymerization (DP) of 4.

### TECHNICAL FIELD

The invention relates to a method for preparing a mixture of poorly-digestible alpha-glucans from a substrate rich in oligosaccharides having a degree of polymerization (DP) of 4. In the present application, this substrate designates a syrup containing oligosaccharides with a content of oligosaccharides having a degree of polymerization (DP) of 4 of at least 40%, preferably of at least 45%, even more preferably of at least 50%.

The invention also relates to a mixture of poorly-digestible alpha-glucans.

The present invention also relates to the use of an alpha-glucanotransferase capable of creating alpha (1,6) glycosidic bonds to reduce the digestibility of a mixture of alpha-glucans.

### PRIOR ART

Dietary fiber has an important role in human nutrition. Among dietary fibers, a distinction is made between soluble fibers, which are soluble in water and have a gelling capacity, and insoluble fibers. Soluble fibers, including branched maltodextrins, are particularly advantageous because they are poorly digestible. Because of this, their incorporation into the diet makes it possible to reduce the glycemic index of a food and to prolong the sensation of satiety. They are also endowed with prebiotic properties on the intestinal flora; in other words, they are capable of selectively promoting the growth of certain bacteria of probiotic type or the activity of the microbiota, by providing a health benefit.

Hitherto, soluble fibers, including branched maltodextrins, were mainly obtained physicochemically.

This is the case in particular with maltodextrin sold by the Applicant company under the brand name NUTRIOSER FM10 as water-soluble fiber.

Other soluble fibers obtained physicochemically exist, such as PROMITOR® sold by the company Tate and Lyl, FIBERSOL® or LITESSE® sold by the company Dupont Nutrition and Biosciences.

Numerous studies have demonstrated that the digestibility properties were directly linked to the percentages of the various types of glycosidic bonds within the soluble fibers.

Indeed, standard maltodextrins are rapidly digestible and are defined as purified and concentrated mixtures of glucose and glucose polymers essentially linked at alpha 1->4 (hereinafter 1->4 or alpha (1,4)) with only 4 to 5% of alpha 1->6 glycosidic bonds (hereinafter 1->6 or alpha (1,6)), of extremely varied molecular weights, completely soluble in water and of low reducing power.

By increasing the percentage of alpha 1->6 or alpha 1->3 bonds, the degree of branching of the maltodextrins is increased, which makes them more resistant to digestion.

The enzymatic approach, which uses enzymes capable of promoting the creation of “branched” type bonds, has numerous advantages, in terms of safety and environmental preservation, and also offers better specificity.

Originally, most enzymatic processes for producing soluble fibers are carried out using sucrose as a substrate for the enzyme, in order to create new bonds. For example, WO2015183714 describes an enzymatic reaction from a mixture of sucrose and alpha-glucan type substrate.

At present, most enzymatic processes use amylomaltases to produce soluble fibers from starch.

It is desirable to obtain enzymatically soluble fibers from the substrate, in the absence of sucrose.

### DETAILED DESCRIPTION OF THE INVENTION

The Applicant company has then found that it is possible, from a syrup rich in oligosaccharides having a degree of polymerization (DP) of 4, to obtain fibers of interest in human and animal nutrition, enzymatically. The Applicant company has thus developed a method that uses a particular enzyme, capable of creating alpha (1,6) bonds from syrup rich in DP4 oligosaccharides.

In a first aspect, the present invention relates to a method for preparing a mixture of alpha-glucans, preferably a mixture of branched maltodextrins, comprising a step of bringing together a substrate and an enzyme, said substrate being a syrup rich in oligosaccharides having a degree of polymerization (DP) of 4 and said enzyme being an alpha-glucanotransferase capable of cleaving alpha (1,4) glycosidic bonds and of creating alpha (1,6) glycosidic bonds.

According to the present invention, the terms “alpha-glucan”, “soluble fiber”, “food soluble fiber” are used interchangeably. They define oligosaccharides composed of at least 3 glucose units linked together by alpha-glycosidic (or alpha-glucosidic) bonds.

The classification of alpha-glucans is mainly based on the measurement of their reducing power, conventionally expressed by the notion of “dextrose equivalent” (“Dextrose Equivalent” or DE). On this particular point, the definition of maltodextrins given in the Monograph Specifications of the Food Chemical Codex specifies that the DE value for a maltodextrin must not exceed 20. Above 20, these are glucose syrups.

Such a DE measurement is however insufficient to accurately represent the molecular distribution of the alpha-glucans. Indeed, the acid hydrolysis of starch, which is totally random, or its enzymatic hydrolysis, which is slightly more ordered, provides mixtures of glucose and glucose polymers that the sole measurement of DE does not make it possible to define with precision, and which comprise molecules of short size, of low DP, as well as molecules of very long size, of high DP.

Measurement of the DE in fact gives only an approximate idea of the average DP of the mixture of glucose and of the constituent glucose polymers of the alpha-glucans and therefore of their number-average molecular mass (Mn). To complete the characterization of the molecular mass distribution of alpha-glucans, it is important to determine another parameter, that of the weight-average molecular mass (Mw).

In practice, (Mn) and (Mw) are determined experimentally by different analysis techniques, such as for example, a measurement method suitable for glucose polymers, which is based on gel permeation chromatography on chromatography columns calibrated with pullulans of known molecular masses.

The Mw/Mn ratio is called the polymolecularity index (PI) and makes it possible to characterize overall the molecular mass distribution of a polymer blend. As a general rule, the molecular mass distribution of standard maltodextrins results in IP values of between 5 and 10.

These various parameters are also the reflection of the alpha-glycosidic bond profile of the alpha-glucans. Indeed, a mixture of standard alpha-glucans has a very high percentage of “linear” alpha (1,4) bonds (greater than 90%) and a low percentage of so-called “branched” (alpha (1,2), alpha (1,3) and alpha (1,6) bonds).

The method according to the present invention makes it possible to reduce the percentage of alpha (1,4) bonds in favor of alpha (1,6) bonds, which has the advantage of reducing the digestibility of the mixture of alpha-glucans obtained by the method.

The mixture of alpha-glucans prepared according to the method of the invention is preferably a mixture of branched maltodextrins.

For the purposes of this invention, branched maltodextrins are understood to be maltodextrins with a higher alpha (1,6) glycosidic bond content than standard maltodextrins.

Standard maltodextrins are defined as purified and concentrated mixtures of glucose and glucose polymers essentially linked at alpha (1,4) with only 4 to 5% of alpha (1,6) glycosidic bonds, of extremely varied molecular weights, completely soluble in water and of low reducing power.

According to one embodiment of the invention, the syrup rich in oligosaccharides having a DP of 4 comprises at least 40%, preferably at least 45%, even more preferably at least 50% of oligosaccharides having a DP of 4.

According to one embodiment of the invention, the syrup rich in oligosaccharides having a DP of 4 has a dextrose equivalent (DE) greater than 20.

According to a preferred embodiment of the invention, the syrup rich in DP4 is a syrup having the characteristics described in Table 1 below.

In a preferred embodiment of the invention, the substrate is present at a concentration of between 50 g/L and 500 g/L, preferably between 100 g/L and 200 g/L, in the reaction medium.

In a preferred embodiment of the invention, the alpha-glucanotransferase capable of cleaving the alpha (1,4) glycosidic bonds and of creating alpha (1,6) glycosidic bonds is the protein having the sequence SEQ ID No: 1 or a protein having at least 90% identity with the protein having the sequence SEQ ID No:1 (hereinafter known as GT #19). Preferably, it is a protein having at least 91%, even more preferably at least 92%, at least 93%, at least 94%, at least 95%, at least 96%, at least 97%, at least 98%, at least 99%, at least 99.5%, at least 99.6%, at least 99.7%, at least 99.8%, at least 99.9% identity with the protein having the sequence SEQ ID No:1. The sequence SEQ ID No:1 corresponds to the Genbank accession number WP 053069107.1.

As shown in the examples, the inventors have demonstrated that enzyme GT #19 is capable of modifying a DP4-rich syrup so as to make it poorly digestible, with a percentage of hydrolysis according to method AOAC2002.02 less than or equal to 45%).

According to one embodiment of the invention, the enzyme is added at a concentration of between 0.01 and 1 mg/ml of reaction medium, preferably between 0.05 and 0.5 mg/mL, even more preferably about 0.1 mg/ml of reaction medium.

According to one embodiment of the invention, the substrate and the enzyme are brought into contact for a period of between 12 and 48 hours, preferably approximately 24 hours

According to one embodiment of the invention, the substrate and the enzyme are brought together at a temperature of between 2° and 40° C., preferably approximately 37° C.

According to one embodiment of the invention, the substrate and the enzyme are brought together at a pH of between 5 and 6.5, preferably between 5.5 and 6 and even more preferably approximately 5.75.

In one embodiment of the invention, the method further comprises a step of enzymatic treatment by an alpha-glucanotransferase capable of cleaving alpha (1,4) glycosidic bonds and of creating alpha (1,3) glycosidic bonds. It may for example be a protein having the sequence SEQ ID No:2 or a protein having at least 90% identity with the protein having the sequence SEQ ID No:2 (hereinafter known as GT #11). Preferably, it is a protein having at least 91%, even more preferably at least 92%, at least 93%, at least 94%, at least 95%, at least 96%, at least 97%, at least 98%, at least 99%, at least 99.5%, at least 99.6%, at least 99.7%, at least 99.8%, at least 99.9% identity with the protein having the sequence SEQ ID No:2. The sequence SEQ ID No: 2 corresponds to the Genbank accession number AOR73699.1.

According to one aspect, the present invention also relates to a mixture of alpha-glucans, preferably a mixture of branched maltodextrins, which can be obtained by the method described hereinbefore.

This mixture of alpha-glucans is characterized by its low digestibility according to the AOAC 2002.02 method. Advantageously, the process according to the invention makes it possible to reduce the hydrolyzable fraction, measured according to the AOAC 2002.02 method, by a factor of at least 2, preferably of at least 2.5, even more preferably of at least 3, relative to the starting substrate.

The AOAC 2002.02 method can in particular be implemented using the “HPAEC-PAD assay” part of the “Starch resistant, K-RSTAR 06/18” kit sold by the company Megazyme® as described in Example 1, part 5 below.

The method according to the present invention makes it possible to increase the percentage of alpha (1,6) bonds by a factor of at least 3, preferably at least 3.5, even more preferably of at least 4, relative to the starting substrate.

The percentage of alpha (1,6) bonds can be measured by the Hakomori method (1964 HAKOMORI A Rapid Permethylation of Glycolipid, and Polysaccharide Catalyzed by Methylsulfinyl Carbanion in Dimethyl Sulfoxide) as described in Example 1, part 8 below or by proton NMR as described in Example 1, part 7 below.

According to one aspect, the present invention relates to a mixture of alpha-glucans, preferably a mixture of branched maltodextrins, characterized in that it has:
-a content of hydrolyzable fibers of less than 55%, preferably less than 50%, even more preferably less than 45%,
and/or at least 20% alpha (1,6) bonds, wherein the fiber content corresponds to the hydrolyzable (that is, non-resistant) fraction according to the AOAC 2002.02 method and the percentage of alpha (1,6) bonds represents the molar percentage of alpha (1,6) bonds relative to the total number of glycosidic bonds, measured by the Hakomori method.

Preferably, the content of hydrolyzable fibers is less than 44%, preferably less than 43%, even more preferably less than 42%, 41%, 40%, 39%, 38%, 37%, 36%, 35%, 34%, 33%, 32%, 31%, 30%.

Preferably, the content of hydrolyzable fibers is greater than 5%, preferably greater than 10%, 11%, 12%, 13%, 14%, 15%, 18%, 19%, 20%, 21%, 22%, 23%, 24%, 25%.

Preferably, the content of hydrolyzable fibers is between 5% and 45%, preferably between 10% and 45%, preferably between 20% and 44%, even more preferably between 30% and 45%.

Preferably, the percentage of alpha (1,6) bonds, is at least 21%, preferably at least 22%, even more preferably at least 23%, at least 24%, at least 25%, at least 26%, at least 27%, at least 28%, at least 29%, at least 30%, at least 31%, at least 32%, at least 33%, at least 34%, at least 35%.

Preferably, the percentage of alpha (1,6) bonds is at most 40%, preferably at most 36%, at most 35%, at most 34%, at most 33%, at most 32%, at most 31%.

Preferably, the percentage of alpha (1,6) bonds is between 20% and 40%, preferably between 20% and 35%, more preferably between 25% and 35%.

Preferably, the percentage of alpha (1,3) bonds is at least 2%, preferably at least 3%.

Preferably, the percentage of alpha (1,3) bonds is at most 6%, preferably at most 5%.

Preferably, the percentage of alpha (1,3) bonds is between 2% and 8%, preferably between 3% and 5%.

Preferably, the percentage of alpha (1,2) bonds is at least 1%, preferably at least 2%.

Preferably, the percentage of alpha (1,2) bonds is at most 8%, preferably at most 6%, at most 5%, at most 4% or at most 3%.

Preferably, the percentage of alpha (1,4) bonds is at most 80%, preferably at most 70%, at most 65%.

Preferably, the percentage of alpha (1,4) bonds is at least 50%, preferably at least 55%, at least 60%.

Preferably, the percentage of alpha (1,4) bonds is between 50% and 80%, preferably between 55% and 70%, more preferably between 55% and 65%.

Preferably, the mixture of alpha-glucans, which is preferably a mixture of branched maltodextrins, is characterized in that it has:
a content of hydrolyzable fibers of less than 55%, preferably less than 50%, even more preferably less than 45%,
and has one or more of the following characteristics:
between 20% and 40% of alpha (1,6) bonds,
between 20% and 40% of alpha (1,3) bonds,
between 2% and 8% of alpha (1,2) bonds,
between 50% and 80% of alpha (1,4) bonds, wherein the fiber content corresponds to the hydrolyzable (that is, non-resistant) fraction according to the AOAC 2002.02 method and the percentage of alpha (1,6), alpha (1,3), alpha (1,2) or alpha (1,4) bonds represents the molar percentage of said type of bonds relative to the total number of glycosidic bonds, measured by the Hakomori method.

Preferably, said content of alpha (1,6), alpha (1,3), alpha (1,2) or alpha (1,4) bonds are such that the sum of their molar percentage is equal to 100%.

The present invention also relates to the use of a mixture of alpha-glucans obtained according to the method described hereinbefore and of a mixture of alpha-glucans having the properties described hereinbefore for the preparation of foods for human or animal nutrition.

Typically, the mixture of alpha-glucans according to the invention can be used to promote intestinal health, blood glucose management, satiety and weight management, and sustained energy release.

Finally, in another aspect, the present invention relates to the use of a glucanotransferase capable of cleaving the alpha (1,4) glycosidic bonds and of creating alpha (1,6) glycosidic bonds in order to reduce the digestibility of a mixture of alpha-glucans, said glucanotransferase having the sequence SEQ ID No: 1 or a protein having at least 90% identity with the protein having the sequence SEQ ID No:1.

In this aspect of the invention, the mixture of alpha-glucans is preferably an oligosaccharide-rich syrup, in particular an oligosaccharide-rich syrup having a DP of 4 as described in the first aspect of the invention.

Preferably, the decrease in digestibility is a decrease by a factor of at least 2, preferably of at least 2.5, even more preferably of at least 3 of the hydrolyzable fraction, measured according to the AOAC 2002.02 method, relative to the starting substrate.

The invention will be better understood with the aid of the following examples, which are intended to be illustrative and non-limiting.

Example 1.: preparation of branched maltodextrins from a syrup rich in DP4: material and methods

### 1. Preparation of a DP4 Substrate Solution

The starting substrate used was a syrup rich in DP4, having the characteristics described in Table 1:

TABLE 1

| | | Measurement | | |
|---|---|---|---|---|
| Criteria | Sub-criteria | method \| | Unit | Value |
| Molecular | Mn | MCL1439 | Da | 615 |
| weight | (pullulan eq) | | | |
| Molecular | Mw | MCL1439 | Da | 1045 |
| weight | (pullulan eq) | | | |
| Carbohydrate | DP1 | MCL190A | Relative % | 2.5 |
| distribution | | | | |
| Carbohydrate | DP2 | MCL190A | Relative % | 6.1 |
| distribution | | | | |
| Carbohydrate | DP3 | MCL190A | Relative % | 7.4 |
| distribution | | | | |
| Carbohydrate | DP4 | MCL190E | Relative % | 55.7 |
| distribution | | | | |
| Carbohydrate | DP5 | MCL190E | Relative % | 2.3 |
| distribution | | | | |
| Carbohydrate | DP6 | MCL190E | Relative % | 2.1 |
| distribution | | | | |
| Carbohydrate | DP7 | MCL190E | Relative % | 2.3 |
| distribution | | | | |
| Carbohydrate | DP8 | MCL190E | Relative % | 5.5 |
| distribution | | | | |
| Carbohydrate | DP9 | MCL190E | Relative % | 1.4 |
| distribution | | | | |
| Carbohydrate | DP10 | MCL190E | Relative % | 1.3 |
| distribution | | | | |
| Carbohydrate | > DP10 | MCL190E | Relative % | 13.4 |
| distribution | | | | |
| Reducing | Dextrose | MCL050B | | 33.4 |
| sugars | equivalent | | | |
| | (DE) | | | |
| Dry material | Loss of | MCL209A | % | 30.2 |
| | mass after | | | |
| | drying | | | |

Various substrate solutions (syrup rich in DP4) in 50 mM sodium acetate buffer, pH 5.75, were prepared at concentrations of 100 g/L, 200 g/L or 400 g/L.

### 2. Production of Recombinant Enzymes.

The following enzymes were recombinantly produced:
Enzyme GT #11: alpha-4,3 glucanotransferase from Lactobacillus fermentum NC2970 having as amino acid sequence the sequence listed in Genbank under the reference AOR73699.1 (SEQ ID No:2).
Enzyme GT #19: glycoside hydrolase GH70 from Lactobacillus mucosae having as amino acid sequence the sequence listed in Genbank under the reference WP_053069107.1. (SEQ ID No: 1)

E. coli BL21 star cells containing plasmid pET-21 a-enzyme no.X (in order to produce various enzymes, including GT #11 and GT #19) were cultured in ZYM-5052 medium containing 1% glycerol and 1% lactose. At the end of the culture, the cells were centrifuged at 6500 g for 10 minutes, the cell pellets resuspended at a DO600 nm of 80 in a 20 mM phosphate buffer, pH=7.4, containing 300 mM of NaCl and 20 mM of imidazole, and the cells were lysed by cold sonication using 4 cycles of 20 seconds at 30% amplitude followed by 4 minutes of rest. The cell debris was separated from the solubilized proteins by centrifugation for 30 minutes at 10,000 g.

### 3. Purifying Enzymes

Purification of the proteins of interest was carried out on Cobalt resin (Invitrogen) loaded with divalent cobalt ions (CO2+), for which the polyhistidine tag has an affinity. Elution was carried out by creating a competition between the polyhistidine tag and increasing concentrations of imidazole. Briefly, 10 to 35 mL of cell extract of E. coli were brought into contact for 1 hour with 1 mL of Cobalt resin equilibrated beforehand with 25 mL of 20 mM Phosphate buffer, pH=7.4, containing 300 mM of NaCl and 20 mM of imidazole. Filtration of the resin on sintered glass allows all of the unbound proteins to be removed. The resin was then washed 5 times with 40 mL of 20 mM Phosphate buffer, pH=7.4, containing 300 mM NaCl and 20 mM imidazole. Finally, elution was carried out with 3 mL of 20 mM Phosphate buffer, pH=7.4, containing 300 mM NaCl and 250 mM imidazole for 5 minutes in order to detach the enzymes of interest. The enzymatic solutions were then dialyzed (10 kDa molecular weight cut-off membrane) against 5 L of 50 mM sodium acetate buffer, pH=5.75 (overnight, 4° C. under stirring) in order to remove the NaCl and imidazole. The various protein solutions were assayed by measuring their absorbance at 280 nm using a 2000 spectrophotometer nanodrop (Thermofisher). The molecular extinction coefficients & were determined using the ProtParam tool application of the ExPASy bioinformatics resource portal site.

### 4. Enzymatic Reactions

The reactions were carried out with 0.1 mg/mL of purified enzyme and dialyzed in the presence of 10%, 20% or 40% of substrate in 50 mM sodium acetate buffer, pH=5.75. The reactions were incubated under stirring for 24 hours at 37° C. The reactions were stopped by heating (95° C. for 5 minutes). Samples were taken at the initial and final times to analyze the specificity of the enzymes using different analytical techniques (HPAEC-PAD, NMR).

### 5. Digestibility Test

The transfer reactions were freeze-dried after freezing at −80° C. for 24 hours. 25 mg of freeze-dried products were taken up in 1 mL of 100 mM sodium maleate buffer containing 30 U of pancreatic «-amylase and 3 U of amyloglucosidase (Starch resistant kit, Megazyme K-STAR 06/18, which implements the AOAC 2002.02 method). The reactions were incubated for 16 hours at 37° C. The products were diluted in water before HPAEC PAD analysis.

### 6. Chromatographic Analyses

The products obtained were analyzed by anion exchange chromatography coupled to a pulsed amperometric detector (HPAEC PAD-HIGH Performance Anion Exchange Chromatography with Pulsed Amperometric Detection). The analyses were carried out on a Thermo ICS6000 system equipped with a CarboPac™ PA100 analytical column (2 mm×250 mm) coupled with a CarboPac™ PA100 guard pre-column (2 mm×50 mm). A gradient of sodium acetate in 150 mM sodium was applied at a flow rate of 0.250 ml·min-1 according to the following profile: 0-5 min, 0 mM; 5-35 min, 0-300 mM; 35-40 min, 300-450 mM; 40-42 min, 450 mM. Detection was carried out using a gold working electrode and a pH Ag/AgCI reference cell. The samples were diluted to a total dry mass of 1 g·1 before injection.

### 7. NMR.

Spectra ¹H, ¹³C and HSQC were recorded on a Bruker Avance 500 MHz equipment at 298 K with a 5 mm Z-gradient H-BB-D BBI probe. The data were acquired and processed using the TopSpin 3 software.

### 8. Hakomori Method

The Hakomori method (1964 HAKOMORI A Rapid Permethylation of Glycolipid, and Polysaccharide Catalyzed by Methylsulfinyl Carbanion in Dimethyl Sulfoxide) makes it possible to chemically characterize the glycosidic bonds by differentiating the free OH groups and the bonded groups. This is a destructive method comprising the steps of methylation, hydrolysis, reduction with NaBD₄, acetylation and analysis by mass spectrometry.

### Example 2.: Preparation of Branched Maltodextrins from a Syrup Rich in DP4: Results

The results of the various enzymatic reactions are presented in Table 2 below, which shows the percentages of alpha-1,6; alpha-1,3 and alpha-1,4 bonds measured by proton NMR or by the Hakomori method and percentage of hydrolysis (AOAC 2002.02) in the reaction products obtained.

TABLE 2

| | | | | | | | | | | % hydrolysis |
|---|---|---|---|---|---|---|---|---|---|---|
| | Concentration | NMR | | | | HAKOMORI | | | | (AOAC |
| Enzyme | g/L | α-1,2 | α-1,3 | α-1,4 | α-1,6 | α-1,2 | α-1,3 | α-1,4 | α-1,6 | 2002.02) |
| RM | 100 | 0% | 0% | 99% | 1% | 2.3% | 2.6% | 93.1% | 2.0% | 78.0% |
| RM | 200 | 0% | 0% | 99% | 1% | 2.3% | 2.6% | 93.1% | 2.0% | 80.0% |
| RM | 400 | 0% | 0% | 99% | 1% | 2.3% | 2.6% | 93.1% | 2.0% | 83.0% |
| GT#11 | 100 | 0% | 10% | 90% | 0% | 2.9% | 10.6% | 82.3% | 4.1% | 48.7% |
| GT#11 | 200 | 0% | 9% | 91% | 1% | 2.7% | 9.3% | 84.4% | 3.7% | 52.9% |
| GT#11 | 400 | 0% | 5% | 92% | 3% | 2.3% | 6.6% | 88.5% | 2.9% | 62.8% |
| GT#19 | 100 | 0% | 0% | 57% | 43% | 2.1% | 4.3% | 62.6% | 30.9% | 31.6% |
| GT#19 | 200 | 0% | 0% | 59% | 41% | 2.2% | 4.3% | 63.0% | 30.5% | 34.8% |
| GT#19 | 400 | 0% | 0% | 65% | 35% | 2.3% | 4.1% | 63.8% | 29.7% | 40.0% |

RM: Raw material, =DP4 syrup

The inventors have demonstrated that enzyme GT #19 is capable of modifying the DP4-rich syrup so as to make it poorly digestible (percentage of hydrolysis according to method AOAC2002.02 less than or equal to 45%).

The product obtained by this enzymatic treatment with enzyme GT #19 contains significantly fewer alpha-1,4 bonds and more alpha-1,6 bonds than the starting point. The number of alpha-1,2 and alpha-1,3 bonds remains unchanged. Enzyme GT #19 is therefore a 4,6-alpha-glucanotransferase.

Conversely, treatment with another GT, enzyme GT #11, leads to a reduction in the percentage of alpha-1,4 bonds and the appearance of alpha-1,3 bonds. The digestibility of the product obtained by treatment with enzyme GT #11 is also reduced (53 and 55%, for initial concentrations of 100 and 200 mg/mL respectively, compared with 84 and 88% for untreated substrates).

Advantageously, the mixture of alpha-glucans according to the present invention also has an interesting in vitro digestibility profile according to the Englyst method.

1. A method for preparing a mixture of alpha-glucans comprising a step of bringing together a substrate and an enzyme, said substrate being a syrup rich in oligosaccharides having a degree of polymerization (DP) of 4 and said enzyme being an alpha-glucanotransferase capable of cleaving alpha (1,4) glycosidic bonds and of creating alpha (1,6) glycosidic bonds.

2. The method according to claim 1, wherein the syrup rich in oligosaccharides having a DP of 4 comprises at least 40%, preferably at least 45%, even more preferably at least 50% of oligosaccharides having a DP of 4.

3. The method according to claim 1 or 2, wherein the syrup rich in oligosaccharides having a DP of 4 has a dextrose equivalent (DE) greater than 20.

4. The method according to any one of the preceding claims, wherein the substrate is at a concentration of between 50 g/L and 500 g/L, preferably between 100 g/L and 200 g/L of reaction medium.

5. The method according to any one of the preceding claims, wherein the alpha-glucanotransferase capable of cleaving the alpha (1,4) glycosidic bonds and of creating alpha (1,6) glycosidic bonds is the protein having the sequence SEQ ID No:1 or a protein having at least 90% identity with the protein having the sequence SEQ ID No:1.

6. The method according to any one of the preceding claims, wherein the enzyme is at a concentration of between 0.01 and 1 mg/ml of reaction medium, preferably between 0.05 and 0.5 mg/mL, even more preferably approximately 0.1 mg/ml of reaction medium.

7. The method according to any one of the preceding claims, characterized in that the substrate and the enzyme are brought together for a period of between 12 and 48 hours, preferably approximately 24 hours and/or at a temperature comprised between 2° and 40° C., preferably approximately 37° C. and/or at a pH of between 5 and 6.5, preferably approximately 5.75.

8. The method according to any one of the preceding claims, characterized in that it further comprises a step of enzymatic treatment by an alpha-glucanotransferase capable of cleaving alpha (1,4) glycosidic bonds and of creating alpha (1,3) glycosidic bonds.

9. The method according to the preceding claim, wherein the alpha-glucanotransferase capable of cleaving the alpha (1,4) glycosidic bonds and of creating alpha (1,3) glycosidic bonds is the protein having the sequence SEQ ID No:2 or a protein having at least 90% identity with the protein having the sequence SEQ ID No:2.

10. A mixture of alpha-glucans capable of being obtained by the method according to any one of the preceding claims.

11. The mixture of alpha-glucans characterized in that it has:
a content of hydrolyzable fibers of less than 55%, preferably less than 50%, even more preferably less than 45%,
and/or at least 20% alpha (1,6) bonds,
wherein the fiber content corresponds to the hydrolyzable fraction according to the AOAC 2002.02 method and the percentage of alpha (1,6) bonds represents the molar percentage of alpha (1,6) bonds relative to the total number of glycosidic bonds, measured by the Hakomori method.

12. Use of a mixture of alpha-glucans according to any one of claim 10 or 11 for preparing food for human or animal nutrition.

13. Use of a glucanotransferase capable of cleaving the alpha (1,4) glycosidic bonds and of creating alpha (1,6) glycosidic bonds in order to reduce the digestibility of a mixture of alpha-glucans, said glucanotransferase having the sequence SEQ ID No:1 or a sequence having at least 90% identity with the protein having the sequence SEQ ID No:1.


SEQUENCE LISTING (2 sequences)

SEQ ID NO: 1 - protein, 881 aa, Lactobacillus mucosae
MKNLVAKPQGNQLNIYNGNTVLKTLGPGTWENMAFAQDSSAINNINGYLSYTGWYRPYGTSQDGKTWYQTTAMDWRPLLMYTWPSKDVQAQFIKYFVNHGYENANYGLTKVSVANLNKDTDATVLNTAAQNLRYVIEQSIATNKGTGKLANDINGFAATVPELSASSELSLQSMPNYKPDESGTVDSDQVIFVNNDSKDQRRGNTSYADSKYRLMNRTINNQTGNDNSDNSPELLVGNDIDNSNPVVQAENLNWEYFLLNYGKLMGYNQDGNFDGFRIDAADNINADVLDQMGQLMNDMYHMKGNPQNANNHLSYNEGYHSGAAQMLNEKGNPQLYMDSGEFYTLENVLGRANNRDNISDLVTNSIVNRQNDVTENEATPNWSFVTNHDQRKNLINRLIIKDHPGIAYIMGSAYKAEYANQAWQEFYADQKKTDKQYAQYNVPAQYAILLSNKDTVPQIYYGDLYNETAQYMQGKSIYYDAITTLMKARKQFVSGGQTMTKLSDNLIASVRYGKGVANANSEGTDSLSRTSGMAVIVGNNPQMAEQTISINMGRAHANEQYRNLLDTTDNGLTYNADGAENPETLTTDDNGILKVTVKGYSNPYVSGYLGVWVPVVSGNQDVTTNAATVSADSNKIFESNAALDSHMIYEDFSLYQPEPTSTENHAYNIIAQNAELFNNLGITDFWMAPAYTPFGMSRYNEGYSMTDRYNLGTNANPTKYGSGEELANAIAALHSAGLKVQEDIVMNQMIGFSGQEAVTVTRTDDRGMQIYVNGKTYANQIYFAYTTGGGNGQETYGGKYLSELQSKYPDLFTTKAISTGVAPDPTTHITKWSAKYENGTSLQNIGIGLAVKLPNGEYAYLRSSDNKSFNTLLPSEISVKL

SEQ ID NO: 2 - protein, 1593 aa, Lactobacillus fermentum
MEFKKHFKLYKSGKQWCIAAVVTFTATLGLVAGNTTVNADTLTTGGSTPQAQVNQEKGITNTSQPASDQGKTSNTGDVRNTVPQPNNSDKTNQYQADTLTTGGSTPQAQVNQEKGITNTSQPASDQGKTSNTGDVRNTVPQPNNSGKTNQSQNGQDQKESAGNDNGSTGNNDQNKGQQTTDWQKNANNQWVYNGKTDQDLKGTQYVQLPTIPDTNVQGNTNWYFVKDGIAQSGVQQWAGTYYYFDPTTYLRVDNDYRQSQWGDWYLFGNDGRILSGVQQWAGTYYYFDPTTYLRVDDDYVTSQWGLKYMFGKDGRIATGLYKWDKNNQWYYFNPTTYLAVTNNYIQANDGHWYLFTADGTAASRVAKWAGTYYYFDPQTHLRVDDNYVQSQWGDWYMFGKDGRIATGLYKWDKNNQWYYFDPTTYLAVTNNYIQANDGHWYLFTADGTAASRVAKWAGTYYYFDPVTHLRVDNNYVQSQWGDWYLFGNDGRILSGVQQWAGTYYYFDPTTYLRVDDDYVTSQWGLKYMFGKDGRIATGLYKWDKNNQWYYFDPTTYLAVTNNYIQANDGHWYLFTADGTAASRVAKWAGTYYYFDPQTHLRVDDNYVQSQWGDWYMFGKDGRIATGLYKWDKNNQWYYFDPVTYLKVTNKWVDGNYYDEDGAQAISKLVTINNRLYYFDDQGKEISNQFRTIHGDKYYFGNDSAAVTGQQTIDGKVYKFSNYGYLLGNRYGKIENGKLNIYSLADHSLIKTVEAGPWENMAYSMDSNSINNIDGYISYTGWYRPYGTSQDGKTWYPTTVADWRPILMYVWPSKDVQVKFIQYFVNHGYENSNYGLTAGSVKDLSENTASINLNEVAQNLRYVIEQHIVAAKSTSQLANDINNFITTIPELSASSELPDESGSGQVIFVNNDNTSYADSKYRLMNRTVNNQTGNDNSDYCPEFVVGNDIDNSNPVVQAENLNWEYFLLNYGKLMGYNQDGNFDGFRIDAADDMDADVLDQIGQLMNDMYHMKGNPQNANNHLSYNEGYGPGAARMLNKKGNPQLFMDARECNTLENVLGRANNRDTISHLVTDSIVNRQNDVTENEATPNWSYVTNHDIRNNLINGLIIKDHPGMGSAYKAEYANQAWQEFYADQKKTDKQYAQYNVPAQYAILLSNKDTVPQIYYGDLYNETAQYMQEKSIYYDAITTLMKARKQFVSGGQTMTKLSDNLIASVRYGKGVANANSEGTDSLSRTSGMAVIVGNNPQMAEQTISINMGRAHANEQYRNLLDTTDNGLTYNADGAENPETLTTDDNGILKVTVKGYSNPYVSGYLGVWVPVASGNQDVTTNAATVSADSNKIFESNAALDSHMIYEDFSMYQPKPTSTENHAYNIIAQNAELFNNLGITDFWMAPAYTQAGTSRYNEGYSVADRYNLGTNANPTKYGSGEELANAIAALHSAGLKVQEDIVMNQMIGLPGQEAVTVTRADNRGMQTYVNGKTYANQMYFAYTTGGGNGQETYGGKYLSELQSKYPDLFTTRAISTGVAPDPTTHITKWSAKYENGTSLQNIGIGLAVKLANGDYAYLNDSNNKAFNTTLPETMSSTDYYANIEDN